(19) United States
(12) Patent Application Publication
(10) Pub. No.: US 2011/0257110 A1
(43) Pub. Date: Oct. 20, 2011

(54) Composition comprising a glycoside of a mono- or diacylglycerol compound and an oil rich in n-3 polyunsaturated fatty acids, a method of producing the composition and use of the composition

(75) Inventors: Arsalan KHARAZMI, Hellerup (DK); Eydbjørg Winther HANSEN, Hornbaek (DK)

(73) Assignee: HYBEN VITAL LICENS APS, Tranekaer (DK)

(21) Appl. No.: 13/091,318

(22) Filed: Apr. 21, 2011

Related U.S. Application Data
(63) Continuation of application No. PCT/DK2009/000227, filed on Oct. 29, 2009.

(30) Foreign Application Priority Data
Oct. 30, 2008 (DK) PA 2008 01492

(51) Int. Cl. A61K 31/7032; A61P 37/06; A61P 17/06; A61P 9/10; A61P 29/00; A61P 3/10

(52) U.S. Cl. 514/25

(57) ABSTRACT

A composition and use of a composition comprising a glycoside of a mono- or diacylglycerol compound and an oil rich in n-3 polyunsaturated fatty acids, e.g. fish oil. The composition may also comprise a rose hip concentrate. The composition is used to treat inflammatory diseases, e.g. joint diseases such as osteoarthritis, or joint pain and stiffness. By administering the composition on a daily basis, a significant reduction in joint pain and stiffness of the affected joints is achieved, which allows individuals suffering from especially joint diseases to resume daily activities.

### CROSS REFERENCE TO RELATED APPLICATIONS

This application is a continuation of PCT International Patent Application no. PCT/DK2009/000227, having an international Filing date of 28 Oct. 2009, claiming priority in Danish patent application no. PA 2008 01492, filed 30 Oct. 2008.

### FIELD OF THE INVENTION

This invention relates to a composition of a glycoside of a mono- or diacylglycerol compound, preferably retrieved from a plant material, and an oil rich in n-3 polyunsaturated fatty acids and the use thereof for the treatment of or prevention of inflammatory diseases such as, but not limited to pain and stiffness in joints associated with e.g. osteoarthritis.

### BACKGROUND

Various rose hip formulations are known. For example, in U.S. Pat. No. 6,024,960, a rose hip formulation for use as an anti-inflammatory natural medicine is described.

In EP1337263 B1 it was shown that a daily administration of a rose hip concentrate and fish oil is used to treat and/or alleviate the symptoms associated with joint diseases such as osteoarthritis, especially joint pain and joint stiffness.

WO 03/043613 A1 relates to the use of a glycoside of a mono- or diacylglycerol compound for the treatment of inflammatory diseases, and more particularly to the compound 3-β-D-galactopyranosyloxy-2-(octadeca-9Z,12Z,15Z-trienoyloxy)propanyl octadeca-9Z,12Z,15Z-trienoate (also known as GOPO, which is a registered trademark) either obtained from natural sources, such as rose hips (the fruits of Rosa Canina) or prepared by total synthesis. The action of this compound was shown to be alleviating chemiluminescence of polymorphonuclear leucocytes and chemotaxis of mononuclear leucocytes.

Moreover, it was discovered in WO 03/043613 A1, that the compounds may be extracted by organic solvents and a bioguided fractionation procedure of rose hips, which were shown to contain the highly active anti-inflammatory agent GOPO. An aqueous formulation containing the highly anti-inflammatory agent was suggested.

Furthermore, WO 2008/003314 A1 describes another method of isolating a glycoside of a mono- or diacylglycerol compound from a plant material, and to products obtained therefrom. In particular, the document relates to the isolation of and products comprising 3-β-D-galactopyranosyloxy-2-(octadeca-9Z,12Z,15Z-trienoyloxy) propanyl octadeca-9Z,12Z,15Z -trienoate, in particular from a rose hip material.

Fish oil is also a known material that has some beneficial effects. In particular, the n-3 polyunsaturated fatty acids (PUFA) of dietary fish oil are known to reduce the level of triglycerides and very low density lipoprotein cholesterol. Dietary supplementation by, for example, 1.0 ml capsules of fish oil containing about 0.3 g of the primary n-3 PUFA, eicosapentaenoic acid (EPA) and docosahexanoic acid (DHA), have been used, though other concentrations of the PUFA's in supplements are available, ranging for example from 20 to 50% by weight.

Osteoarthritis is characterized by an erosion of articulated cartilage which becomes soft, frayed and thinned with eburnation of subchcondral bone and outgrowths of marginal osteophytes. Pain and loss of function is the result. This is more common in older persons and is considered a degenerative joint disease which mainly affects the weight bearing joints such as the hips and knees.

While inflammation is one symptom of arthritis, the pain and stiffness of the joints are particularly debilitating as this physically inhibits activity and lessens the motivation for daily activities, as well as causes sleeplessness, and results in an overall negative impact on the general well being of an individual, as one susceptible to such pain and joint stiffening must generally refrain from normal daily activities such as walking, entering a vehicle, etc.

However, glycosides of a mono- or diacylglycerol compound, and especially GOPO, have shown to lack stability in aqueous solutions as described in WO03/43613, and tend to degrade during storage. This leads to a decrease in the concentration of the active ingredient in the composition during storage and difficulties in determining whether the dose given to the patient is correct.

When glycosides of a mono- or diacylglycerol compound, and especially GOPO, have shown to lack stability in aqueous solutions it becomes impossible to determine whether the patient is given the correct dose of the anti-inflammatory ingredient, unless the concentration thereof is determined shortly before administering the aqueous composition described in WO03/43613.

### SUMMARY OF THE INVENTION

It is an object of the present invention to provide an improved composition for the treatment of symptoms associated with inflammatory diseases.

It is a further object of the present invention to provide an improved composition for the prophylaxis of inflammatory diseases.

It is a further object of the present invention to provide an improved composition based on natural products to substantially avoid side effects associated with traditional drugs used to treat inflammatory diseases such as joint pain and stiffness related to arthritis, e.g. osteoarthritis.

It is a further object of the present invention to provide a composition which shows a good stability and/or reduced tendency to oxidative degeneration of the active anti-inflammatory agent or agents.

It is yet another object to provide a method of treating and alleviating the symptoms of inflammatory conditions, such as, but not limited to hepatitis, meningitis, rheumatoid arthritis, inflammatory bowl diseases, such as Crohns disease, allergic syndromes, diabetes, congestive heart disease, psoriatic, reactive or osteo-arthritis or other arthritides or joint diseases, multiple sclerosis, atherosclerosis, sepsis/septic shock, dermal inflammation, graft rejection, and inflammation secondary to chemotherapy or radiotherapy of neoplastic diseases.

It is yet another object to provide a method of treating and alleviating the symptoms, e.g. pain and joint stiffness, associated with joint diseases, such as arthritis, osteo-arthrosis etc.

These and other objects of the present invention are achieved by a composition comprising a glycoside of a mono- or diacylglycerol compound and an oil rich in n-3 polyunsaturated fatty acids wherein the glycoside of a mono- or diacylglycerol compound is selected from 3-β-D-galactopyranosyloxy-2-(octadeca-9Z,12Z,15Z-trienoyloxy)propanyl octadeca-9Z,12Z,15Z-trienoate, 3-β-D-glucopyranosyloxy-2-(octadeca-9Z,12Z,15Z-trienoyloxy)propanyl octadeca-9Z,12Z,15Z-trienoate, 3-α-D-galactopyranosyloxy-2-(octadeca-9Z,12Z,15Z-trienoyloxy)propanyl octadeca-9Z,12Z,15Z-trienoate, and 3-α-D-glucopyranosyloxy-2-(octadeca-9Z,12Z,15Z-trienoyloxy)propanyl octadeca-9Z,12Z,15Z-trienoate, 6-O-(α-D-galactopyranosyl)-3-β-D-galactopyranosyloxy-2-(octadeca-9Z,12Z,15Z-trienoyloxy)propanyl octadeca-9Z,12Z,15Z-trienoate and 6-O-(α-D-galactopyranosyl)-3-α-D-galactopyranosyloxy-2-(octadeca-9Z,12Z,15Z-trienoyloxy)propanyl octadeca-9Z,12Z,15Z-trienoate and mixtures thereof, and wherein the oil rich in n-3 polyunsaturated fatty acids is selected from a group comprising fish oil, shark oil, whale oil, seal oil, linseed oil, chia seed oil, kiwifruit seed oil, perilla seed oil, cowberry seed oil and mixtures thereof.

Based on the knowledge that the glycosides of mono- or diacyl glycerol compounds possess anti-inflammatory activity, the inventors have developed a composition based on a glycoside of a mono- or diacylglycerol compound and an oil rich in n-3 polyunsaturated fatty acids.

Using the combination, a surprising improvement in pain relief and reduction in joint stiffness is achieved with a consequent improvement in overall well being.

The composition may preferably further comprise a rose hip concentrate and optionally other ingredients, e.g. antioxidants.

The composition according to the invention comprises GOPO and a n-3 PUFA rich oil, especially fish oil, shark oil, seal oil, whale oil or other sea living mammals or animals, and linseed oil, rapeseed oil, chia seed oil, kiwifruit seed oil, perilla seed oil, cowberry seed oil and mixtures thereof. The composition shows improved stability of GOPO compared to the aqueous solution described in WO 03/043613 A1, without compromising on the anti-inflammatory synergistic effect related to especially joint diseases of the rose hip extract and the fish oil described in EP 1337263 B1.

Since the compositions according to the invention show an improved stability of GOPO, which leads to a reduction of the degradation of GOPO during storage of the composition, it is easier to determine whether the patients are given the correct dose of GOPO in order to achieve the anti inflammatory effect in the patients in comparison with the known less stable aqueous solution.

Further, it is possible to reduce the amount of rose hip extract or increase the amount of the active anti-inflammatory agent such as GOPO in a composition depending on the amounts needed in order to optimise the composition when used for the treatment of one or more of the diseases mentioned above.

Furthermore, it is easier to standardise the composition according to the invention comprising the glycosides of mono- or diacylglycerol compounds and the oil rich in n-3 PUFA, e.g. fish oil, compared to standardising a composition containing fish oil and rose hip extract alone.

### DETAILED DESCRIPTION OF THE INVENTION

The term “glycosides of mono- or diacylglycerol” and similar terms are intended to mean a class of glycosides of mono- or diacylglycerols (as well as ethers), such as those which may be isolated from plants e.g. as illustrated by the methods described in WO 2008/003314 A1 or WO 03/043613 A1, and which are not esters of eicosapentaenoic acid. The “glycoside” part is typically a pentose, hexose or heptose, in particular hexoses such as galactose and glucose, e.g. galactose, but may also be di- and oligosaccharides containing two or more sugar moieties in combination, in particular diglycosides such as digalactosides and diglucosides, e.g. 6-O-(α-D-galactopyranosyl)-α-D-galactopyranose.

In the present context, the term “glycosides of mono- or diacylglycerol product” relates to a product obtained from a plant material comprising a glycoside of mono- or diacylglycerol compound. The glycosides of mono- or diacylglycerol product may be obtained from the plant material by isolation by means of chromatography, microfiltration, filtration, centrifugation, extraction or any combination thereof.

In an embodiment of the present invention, the glycosides of mono- or diacylglycerol compound may have the formula I:

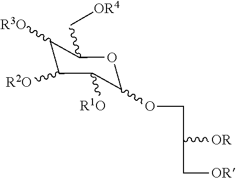

wherein R and R′ are independently selected from hydrogen, C10-24 alkyl, and C10-24 acyl, said alkyl and acyl groups having 0 to 5 unsaturated bonds, and R1, R2, R3 and R4 are independently selected from hydrogen and glycoside moieties; with the first proviso that not both of R and R′ are hydrogen, and with the second proviso that none of R and R′ is eicosapentaenoyl.

The “wavy bonds” in the formulae presented herein are intended to mean that the carbon on which the substituent in question is positioned may be in the (R) or (S) configuration. In the “sugar” moiety (glycoside) the two different configurations are sometimes designated α and β. A particular interesting combination is glucose or galactose in the β-pyranose form.

Although the “sugar” moiety in the formulae presented herein is drawn in the pyranose form, it will be understood that the anti-inflammatory agent may also be present in the furanose form (or a mixture of the pyranose and furanose forms) as a solid and in solution.

In the present context, the term “C10-24 alkyl” is intended to mean a linear or branched hydrocarbon group having 10 to 24 carbon atoms, e.g., decyl, undecyl, dodecyl, tetradecyl, hexadecyl, octadecyl, nonadecyl, eicodecyl, etc.

In the present context, the term “C10-24 acyl” is intended to mean a linear or branched hydrocarbon group having 10 to 24 carbon atoms, wherein the first carbon of the group is a carbonyl (C9-23 alkyl-C(═O)—), i.e. a fatty acid residue having 10 to 24 carbon atoms. Examples thereof are the residues of lauric acid (C12), myristic acid (C14), palmitic acid (C16), stearic acid (C18), etc.

The alkyl and acyl groups may have 0 to 5 unsaturated bonds such as double or triple bonds, in particular double bonds. Examples of acyl groups having one or more unsaturated double bonds are the residues of palitoleic acid (C16:1), oleic acid (C18:1), linoleic acid (C18:2), linolenic acid (C18:3), arachidonic acid (C20:3), retinoic acid (C20:5), etc.

More specifically, the glycosides of mono- or diesters (or ethers) of glycerol are diesters, diethers, or monoether-monoesters, i.e. R and R′ are independently selected from C10-24 alkyl and C10-24 acyl.

The currently most preferred glycosides are glycosides of diesters, i.e. R and R′ are independently C10-24 acyl. In all instances, the alkyl and acyl groups have 0 to 5 unsaturated bonds.

With respect to the degree of saturation of the alkyl and acyl groups, it is currently believed that any alkyl and acyl groups having 0 to 4 unsaturated bonds, such as 1-3 unsaturated bonds, e.g. 2 or 3 unsaturated bonds, in particular 3 unsaturated bonds, are the most suitable ones as R and R′.

Also, it is currently believed that any unsaturated bonds preferably are double bonds.

This being said, it is envisaged that particularly interesting anti-inflammatory agents are those where R and R′ are both C16-20 acyl having 1 to 3 double bonds, such as C18 acyl having 3 double bonds, in particular where the “sugar” moiety is glucose or galactose, in particular galactose.

As mentioned above, R1, R2, R3 and R4 are independently selected from hydrogen and glycoside moieties, preferably at the most only one of R1, R2, R3 and R4 is a glycoside moiety. The latter embodiment relates to compounds that are often found in vegetable sources along with compounds where all of R1, R2, R3 and R4 are hydrogen. In some interesting embodiments, all of R1, R2, R3 and R4 are hydrogen.

The term “glycoside moieties” is intended to mean a mono- or disaccharide moiety, e.g. derived from O-galactopyranose, O-glucopyranose, O-galactopyranosylgalactopyranose, O-glucopyranosylgalactopyranose, O-galactopyranosylglucopyranose and O-glucopyranosylglucopyranose.

Also, it is envisaged that the glycoside of a mono- or diacylglycerol compound preferably has the formula II:

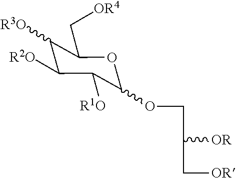

wherein R, R′, R1, R2, R3 and R4 all are as defined above.

More specific examples of the glycoside of a mono- or diacylglycerol compound of particular interest are those selected from β-D-galactopyranosyl derivatives, α-D-galactopyranosyl derivatives, β-D-glucopyranosyl derivatives, and α-D-glucopyranosyl derivatives, such as β-D-galactopyranosyl and β-O-(α-D-galactopyranosyl)-β-D-galactopyranosyl derivatives.

Even more specific examples of the glycoside of a mono- or diacylglycerol compounds are 3-β-D-galactopyranosyloxy-2-(octadeca-9Z,12Z,15Z-trienoyloxy)propanyl octadeca-9Z,12Z,15Z-trienoate, 3-β-D-glucopyranosyloxy-2-(octadeca-9Z,12Z,15Z-trienoyloxy)propanyl octadeca-9Z,12Z,15Z-trienoate, 3-α-D-galactopyranosyloxy-2-(octadeca-9Z,12Z,15Z-trienoyloxy)propanyl octadeca-9Z,12Z,15Z-trienoate, 3-α-D-glucopyranosyloxy-2-(octadeca-9Z,12Z,15Z-trienoyloxy)propanyl octadeca-9Z,12Z,15Z-trienoate, 6-O-(α-D-galactopyranosyl)-3-β-D-galactopyranosyloxy-2-(octadeca-9Z,12Z,15Z-trienoyloxy)propanyl octadeca-9Z,12Z,15Z-trienoate and 6-O-(α-D-galactopyranosyl)-3-α-D-galactopyranosyloxy-2-(octadeca-9Z,12Z,15Z-trienoyloxy)propanyl octadeca-9Z,12Z,15Z-trienoate, such as 3-β-D-galactopyranosyloxy-2-(octadeca-9Z,12Z,15Z-trienoyloxy)propanyl octadeca-9Z,12Z,15Z-trienoate or 3-β-D-glucopyranosyloxy-2-(octadeca-9Z,12Z,15Z-trienoyloxy)propanyl octadeca-9Z,12Z,15Z-trienoate, e.g. 3-β-D-galactopyranosyloxy-2-(octadeca-9Z,12Z,15Z-trienoyloxy)propanyl octadeca-9Z,12Z,15Z-trienoate, which may be isolated from rose-hips.

The most preferred galactolipid is e.g. 3-β-D-galacto-pyranosyloxy-2-(octadeca-9Z, 12Z, 15Z-trienoyloxy)propanyloctadeca-9Z,12Z, 15Z-trienoate (GOPO).

The glycosides of mono- or diacylglycerol compounds, especially GOPO, as mentioned above, are isolated from plant material such as rose hips. The rose hip is preferably obtained from wild rose bushes, in particular the rose hip is selected from the group consisting of Rosa canina (“dog rose hip”), Rosa gallica, Rosa condita, Rosa rugosa, Rosa hugonis, Rosa nitida, Rosa pendulina, Rosa pimpinellifolia, and Rosa sericea.

The plant material is not limited to rose hip only. It might consist of any plant material containing a glycoside of mono- or diacylglycerol compounds, and especially galactolipids, such as fruit, vegetables or cereals, where the fruit, vegetable or cereal is preferably selected from the group consisting of olive (e.g. Olea europaea), alfalfa (e.g. Mediargo sativa L.), soya bean (e.g. Glycine max), potato (e.g. Solanum Turerosum L.), pepper (e.g. Capsicum annuum L.), oat (e.g. Avena sativa), wall cress (e.g. Arabidopsis thaliana), Petunia hybrida, lyme grass (e.g. Elymus arenarius), broom (e.g. Sarothamnus scoparius), coltsfoot (e.g. Tussilago farfara), chenopodiaceae, seakale (e.g. Crambe maritima), sloe (e.g. Prunus spinosa), eryngo (e.g, Eryngium), sea purslane (e.g. Honckenya peploides), blackberry, mountain ash (e.g. Sorbus aucuparia), service tree (e.g. Sorbus domestica), sea buckthorn (e.g. Hippophaë rhamnoides), hemp agrimony (e.g. Eupatorium cannabinum), cucumber (e.g. M. charantia or M. rustrata), Catharanthus roseus, yew (e.g. Taxus baccata), mistletoe (e.g. Viscum album), horsetails (e.g. Equisetum arvense), meadowsweet (e.g. Filipendula ulmaria), dropwort (e.g. F. hexapetala), Ephidera (e.g. E. sp.), reed (e.g. Phragmites communis), ground ivy (e.g. Glechoma hederacea), male fern (e.g. Lastrea filix mas), shield fern (e.g. Dryopteris) and lady's mantle (e.g. Alchemilla vulgaris); seaweed, preferably, the seaweed is selected from the group consisting of Anfeltia tobuchiensis (Rhodophyta), Laminaria japonica, Sargassum pallidum (Phaeophyta), Ulva fenestrate (Chlorophyta), Zostera marina (Embriophyta), sea wrack (Fucus vesiculosus), green alga (Chlorella vulgaris), Cyanobacteria (e.g. Phormidium tenue) and Okinawan Marine Sponge (Phyllospongia Foliascens).

The oil ingredient is preferably an oil naturally rich in n-3 PUFA's, and the preferred oils have a naturally high content of n-3 polyunsaturated fatty acids, especially a-linolenic acid (ALA), eicosapentaenoic acid (EPA) and/or docosahexaenoic acid (DHA). The oils rich in n-3 PUFA's are normally of animal or vegetable origin. The oils rich in n-3 PUFA's of animal origin are seal oil (family: Phocidae), whale oil (family: Cetacea) or other sea living mammals or animals and fish oil originating from oily fish species, e.g. cold water fish such as codfish (especially codfish liver oil), salmon, herring, mackerel, sardines, sand lances/sand eels or anchovies, although other species of oily fish, e.g. tuna or shark may be used. Examples of n-3 PUFA's rich oils of vegetable origin are linseed oil (Linum usitatissimum), rapeseed oil (e.g. Brassica napus L. or B. campestris L), chia seed oil (Salvia hispanica), kiwifruit seed oil (Actinidia chinensis), perilla seed oil (Perilla frutescens), cowberry/lingonberry seed oil (Vaccinium vitis-idaea) and mixtures thereof.

The oil ingredient, which is a natural substance, may vary in its concentration of n-3 PUFA's. Thus, the amount constituting a unit dose may vary in relation to the concentration of the fatty acids. For example, fish oil, which is a preferred n-3 PUFA rich oil, may contain from 1 to 50% by weight of the n-3 PUFA's, particularly, eicosapentaenoic acid and docosahexaenoic acid, typically measured as triglycerides. The lower the concentration of n-3 PUFA's, the higher unit daily dosage should be administered, i.e., five capsules at 10% would be equivalent to one capsule at 50%.

This may, of course, vary with the type of fish used to obtain the fish oil and the processing used to produce the fish oil component, which techniques are conventional in the art. For example, cod fish oil may have about 20% of the n-3 PUFA's while other species may have more or less and this amount may be further concentrated using appropriate conventional processing steps. The most common fish oil supplement is in the form of 1.0 ml capsules, containing about 0.3 g of PUFA's per capsule, though capsules containing up to 50% PUFA's are commercially available.

The fish oil may be obtained in a dry powdered form. For example, a dry powder containing 25% by weight of fish oil is commercially available, one such powder containing 25.7% n-3 18:22 fish oil, having from about 41.0 to 42.2 mg/g EPA as triglycerides and from about 27.0 to 28.8 mg/g DHA calculated as triglycerides. Such a material is well suited for use in accordance with the present invention.

The composition may also contain rose hip extract, which is a naturally occurring source for the glycoside of mono- or diacylglycerol compounds mentioned above. The rose hip is preferably obtained from wild rose bushes, in particular the rose hip is selected from the group consisting of Rosa canina (“dog rose hip”), Rosa gallica, Rosa condita, Rosa rugosa, Rosa hugonis, Rosa nitida, Rosa pendulina, Rosa pimpinellifolia, and Rosa sericea.

Preferably, the rose hip extract is in powdered form and may be pelletized or placed in capsules with a physiologically acceptable carrier for formulation into unit dosages.

A unit dosage comprises a therapeutically effective daily amount of the glycosides of mono- or diacylglycerol compounds, the oil rich in n-3 PUFA's and optionally the rose hip extract, which may be taken as a single daily administration or by multiple small doses taken over the course of a day.

As mentioned above, the composition according to the invention can be formulated for delivery via various routes of administration. Oral administration is preferred for ease of use. A unit dosage can comprise a therapeutically effective amount of the combination of a glycoside of a mono- or diacylglycerol compound and the oil rich in n-3 PUFA's for a single daily administration (e.g. orally or by feeding tube in an enteral diet for example), or be formulated to provide multiple doses per day. A unit dosage will depend on many factors, including age, condition, and disease state, but in any event, the entire daily dosage will be that which is physiologically acceptable to the individual and may be administered daily over a prolonged period of time, as joint diseases are normally chronic illnesses.

The dose of the anti-inflammatory agent, which would be effective in the treatment of the inflammatory condition, in particular arthritis and osteoarthrosis, and relief of the symptoms associated therewith, is currently under investigation. Based on previous results using rose hip extracts for the treatment of arthritis and osteoarthrosis, it is believed that a dosage will be in the range of 0.00150 mg/kg body weight per day, such as 0.005-20 mg/kg body weight per day (mg/kg/day) of the isolated glycoside of mono- or diacylglycerol, especially but not limited to GOPO.

A similar to lesser dose rate could be administered on a daily basis as a prophylactic dose.

In a further embodiment of the present invention, the medicament may be in the form of a daily dose form applicable for administration of it in a dose from about 0.001 to about 50 mg/kg body weight per day and preferably 0.005-20 mg/kg body weight per day or 0.01-500 mg per day of the isolated glycoside of mono- or diacylglycerol, such as GOPO in unitary or multiple doses, the user taking e.g. from one to four capsules per day.

A unit daily dose of n-3 PUFA rich oil, such as fish oil, in accordance with the present invention should be in the range of about 500-3600, more preferably 1300-2600 mg of the n-3 PUFA containing fish oil per day in either a single dose or in multiple smaller doses, so as to provide from about 0.1 to 1.8 g PUFA's per day, more preferably about 0.1 to 0.8 g PUFA's per day.

A preferred unit dosage of the optionally added rose hip extract ingredient will be from about 0.02 to about 0.3 g/kg body weight per day or about 1.5 to 30 g per day. About 2.5 to 15 g per day are preferred, and about 2.5 to 6.0 g, such as 5 g, are most preferred.

A similar to lesser dose rate could be administered on a daily basis e.g. as a prophylactic dose. A preferred unit dosage of the optionally added rose hip extract ingredient will be from about 0.01 to about 0.3 g/kg body weight per day or about 0.75 to 30 g per day. About 0.75 to 6 g per day are preferred, and about 1.0 to 3.0 g are most preferred as a lesser dose.

The fish oil and the rose hip extract both contain naturally occurring anti-oxidants, for example Vitamin E (present in fish oil) and vitamin C (present in rose hip extract). The inventors have found that a composition containing these naturally occurring antioxidants, and especially the Vitamin E in the fish oil optionally in combination with vitamin C in a rose hip extract, appear to reduce degradation of the glycerides of mono- or diacylglycerol compounds such as GOPO in the composition. Therefore, it is normally not necessary to add additional antioxidants if fish oil is used as n-3 PUFA rich oil in the composition, which optionally also contains rose hip extract.

If one or more of the ingredients, i.e. the n-3 PUFA rich oil and/or the rose hip extract, do not contain a sufficient concentration of the naturally occurring antioxidants mentioned above, additional antioxidants may be added to the composition. The preferred supplementary anti-oxidants are e.g. an antioxidant selected from Vitamin C and derivatives thereof, Vitamin E, flavonoides, phenolic acids such as methyl, ethyl or n-propyl p-hydroxybenzoate, carotenes, butylated hydroxyanisoles, butylated hydroxytoluenes, nordihydroguaiaretic acid, etc. and mixtures thereof. This is particularly relevant when the anti-inflammatory agent comprises one or more unsaturated bonds such as double bonds, which renders the compound susceptible to oxidative degradation.

Thus, a preferred composition contains about 500-3600, more preferably 1300-2600 mg of the n-3 PUFA containing fish oil and 0.001 to about 50 mg/kg/day, corresponding to about 0.1 g-1.8 g PUFA's per day, more preferably about 0.1 to 0.8 g PUFA's per day, and preferably 0.005-20 mg/kg body weight per day or 0.01-500 mg of GOPO in a daily unitary dose or multiple doses, the user taking e.g. from one to four capsules per day.

Another preferred composition contains about 500-3600, more preferably 1300-2600 mg of the n-3 PUFA containing fish oil and 0.001 to about 50 mg/kg/day, corresponding to about 0.1 g-1.8 g PUFA's per day, more preferably about 0.1 to 0.8 g PUFA's per day, and preferably 0.005-20 mg/kg body weight per day or 0.01-500 mg of GOPO and a rose hip extract of about 2.5 to 15 g per day and preferably about 2.5 to 6.0 g (corresponding to a daily dose of 0.02 to about 0.3 g/kg body weight per day of rose hip extract) in a daily unitary dose or multiple doses, the user taking e.g. from one to four capsules per day.

Another preferred composition, e.g. for prophylactic use, contains less rose-hip concentrate. This composition contains about 500-3600, more preferably 1300-2600 mg of the n-3 PUFA containing fish oil, corresponding to about 0.1 g-1.8 g PUFA's per day, more preferably about 0.1 to 0.8 g PUFA's per day, and 0.001 to about 50 mg/kg/day and preferably 0.005-20 mg/kg body weight per day or 0.01-500 mg of GOPO and rose hip extract ingredient will be from about 0.01 to about 0.3 g/kg body weight per day or about 0.75 to 30 g per day. About 0.75 g to 6 g per day are preferred, and about 1.0 to 3.0 g are most preferred (corresponding to a daily dose of 0.01 to about 0.3 g/kg body weight per day of rose-hip extract) in a daily unitary dose or multiple doses. Due to the reduced amount of rose hip extract in the unitary daily dose the volume thereof will be reduced and the user takes a reduced number of capsules per day, compared to the above-mentioned composition.

The composition may be prepared as a solid powder using a dry powdered form of the oil rich in n-3 PUFA's, e.g. fish oil, as mentioned above in combination with solid GOPO. If present, the rose hip extract may also be added as a solid powder. The rose hip extract powder may optionally be combined with GOPO, thus producing a GOPO enriched rose hip extract, before mixing with the fish oil powder. It is especially preferred to use a powdered dry form of fish oil or other n-3 PUFA rich oil, if GOPO and optionally rose hip extract are present as solids. This improves the stability of the product even further. The solids are also easier to transport in bulk and are more easily processed into the final product such as tablets or capsules. If the oil rich in n-3 PUFA's, e.g. fish oil, is liquid, the composition may be prepared as a suspension of solid glycerides of mono- or diacylglycerol compounds such as GOPO in the oil rich in n-3 PUFA's. If solid rose hip extract is present in the composition, it may be enriched with the glycerides of mono- or diacylglycerol compounds such as GOPO, before mixing with or suspension in the oil or powder rich in n-3 PUFA's. Alternatively, the glycerides of mono- or diacylglycerol compounds such as GOPO, which is soluble in water, may be added to the oil rich in n-3 PUFA's as an aqueous solution and thus forms an emulsion using conventional physiologically accepted emulsifiers.

In either instance, the composition according to the invention may be manufactured into tablets, capsules, caplets, elixirs, enteral formulations or be incorporated into slow release carriers, where the solid composition as described above, is particularly suitable for being incorporated into tablets, capsules, caplets and slow release carriers.

The composition may further comprise conventional physiologically acceptable carriers and/or adjuvants. Examples of other physiologically acceptable carriers would also include other oils, oil emulsions, alcohol, etc.

Furthermore, the composition may also contain one or more other additional ingredients, which are used in the prophylaxis and/or treatment of e.g. joint diseases or other inflammatory conditions than those mentioned above, e.g. glucosamine, turmeric and/or ginger root extracts and optionally Vitamin D in combination with Calcium etc. The composition according to the invention may be used for the prevention, treatment or alleviation of inflammation, whether caused by illness or medical conditions, such as viral or bacterial diseases (commonly termed “inflammatory conditions”). “Inflammation” is defined in Stedman's Medical Dictionary, 26th Edition as “a fundamental pathologic process consisting of a dynamic mixture of cytological and chemical reactions that occur in the affected blood vessels and adjacent tissue in response to injury or abnormal stimulation caused by physical, chemical or biological agent, including the local reactions and resulting morphologic changes, the destruction of removal of the injurious material, and the responses that lead to repair and healing”. Examples of relevant inflammatory conditions are hepatitis, meningitis, rheumatoid arthritis, inflammatory bowl diseases such as Crohn's disease, allergic syndromes, diabetes, congestive heart disease, joint diseases, psoriatic, reactive or osteo-arthritis or other arthritides such as osteoarthrosis, multiple sclerosis, atherosclerosis, sepsis/septic shock, dermal inflammation, graft rejection, and inflammation secondary to chemotherapy or radiotherapy of neoplastic disease.

The present invention is presently believed to be particularly suitable for the treatment of arthritis and/or osteoarthrosis.

Administration may proceed by oral, buccal, parenteral, topical, rectal, transdermal or intranasal administration, though oral administration is preferred.

The term “mammal” is intended to include larger mammals such as humans as well as domestic or farm animals such as dogs, cats, horses, sheep, pigs, cattle, etc. Among these mammals, humans are particularly interesting subjects to benefit from the invention.

Tests have shown that the combination of rose hip and fish oil is superior to rose hip alone, in alleviating pain and stiffness of the hip and knee. In addition, energy, general well being, motivation for daily activities and sleep improved while on the combination of fish oil and rose hip.

While it was recognized that fish oil to some extent reduced pain and stiffness in patients with joint disease, the salient finding is that rose hip combined with fish oil is proven to alleviate pain and stiffness of the hip and knee, much better than fish oil combined with placebo or rose hip alone. Rose hip significantly adds to the beneficial effect of fish oil on stiffness and pain of the hip and knee. The combination of fish oil and rose-hip improves energy, general well being, motivation for daily activities and sleep compared to fish oil and placebo, and finally the combination also significantly reduces pain.

The tests indicated that the combination of rose hip and fish oil is superior to fish oil alone regarding alleviation of pain and stiffness of the hip and knee. In addition, energy, general well-being, motivation for daily activities and sleep improved while on the combination of fish oil and rose hip.

While it is not completely clear as to why the combination achieves such superior results at such relatively low doses, it is believed that arachidonic acid in the membranes of different cell types may be a key in the production of eicosanoids such as prostaglandin, thromboxane and leucrotriene, which are all essential in the development of cell injury and pain in diseases including rheumatoid arthritis and osteoarthrosis.

Intake of fish oil rich in n-3 PUFA's is believed to result in a decrease in cell membrane arachidonic acid level and a concomitant decrease in the synthesis of eicosanoids (prostaglandins, thromboxanes and leucotriens) from arachidonic acid.

One of the mechanisms behind rheumatoid and osteoarthritic pain and stiffness, is a liberation of cytotoxic agents from neutrophils, which results in the destruction of tissue and the formation of oedema, resulting in pain. The number of neutrophils present influences the amount of tissue destructive components liberated. The migration (chemotaxis) of neutrophils as well as the ability of these leucocytes to liberate cytotoxic agents are therefore essential to the formation of pain and also to the degree of tissue destruction locally.

Eicosanoids of interest to neutrophile function are the leucotriens. Intake of fish oil may decease the production of leucotriens and inhibit neutrophil function. In that way fish oil may act as an anti-inflammatory agent.

In addition, fish oil may inhibit the proliferation of another group of white blood cells, the lymphocytes, as cytotoxic lymphocyte activity has been reported to be significantly diminished after intake of fish oil.

Mechanisms behind the impact of fish oil on pain and stiffness in rheumatoid arthritis and osteoarthrosis therefore may be:

1) inhibition of the production of eicosanoids, e.g., leucotriens, which modify neutrophil function, and

2) inhibition of cell injury by inhibiting cytotoxic activity of neutrophils and lymphocytes.

Rose hip powder has also been tested in vitro using a modified boyden chamber. After the addition of even low doses of rose hip, neutrophil chemotaxis towards the chemotactic peptide f-Met-Leu-Phe and Zymosan was markedly reduced. Moreover, the oxidative burst from neutrophils, when using chemiluminescence, was abolished, indicating antioxidative properties. When chemotaxis and oxidative burst response were tested in humans taking rose hip powder, neutrophil chemotaxis and oxidative burst response significantly declined using even low concentrations of rose hip. It was also shown that in patients suffering from osteoarthrosis, pain significantly declined after four weeks of rose hip treatment. The basic mechanisms behind this reduction in pain and stiffness might well be the reduction in migration of neutrophils (resulting in a lessening of the amount of cytotoxicity liberated) and the reduction in neutrophil oxidative burst response, resulting in less cell destruction. A cell preserving capacity of rose hip was also shown in a study on red cells which sustained more stress before membrane disintegrated in human volunteers treated with rose hip for four weeks.

It has also been demonstrated that glycosides of mono- or diacylglycerol compounds, especially GOPO extracted from rose hips, at fairly low concentrations inhibited the chemiluminescence of human peripheral blood leukocytes. Chemiluminescence is a measure of oxidative burst response. This indicates that the isolated compound exhibits anti-oxidant activity. As the actual tissue damage caused by inflammatory cells such as PMN's and monocytes/macrophages, through the release of proteolytic and hydrolytic enzymes as well as toxic reactive oxygen radicals are activated in the tissue and joints, the isolated compound should be a potent inhibitor of the oxidative burst response of the human peripheral blood polymorphonuclear leukocytes, the most important and abundant inflammatory cells. It is therefore believed that the glycosides of mono- or diacylglycerol compounds, especially GOPO, are responsible for the anti inflammatory effects of rose hip extracts.

As fish oil inhibits the production of eicosanoids such as prostaglandins, thromboxane and leucotriens, it is likely to also inhibit platelet aggregation. Platelet aggregation was not inhibited when rose hip was given to healthy volunteers, suggesting that the arachidonic acid pathway was not inhibited by rose hip. It is therefore believed that the inhibitory effect of neutrophil chemotaxis observed using fish oil and rose hip or GOPO have different pathways (fish oil uses the arachidonic pathway and rose hip uses an alternative pathway not yet fully elucidated). The impact on migration of neutrophils might therefore be synergistic.

In addition, fish oil affects neutrophil and lymphocyte mediated cytotoxicity. Rose hip and GOPO have not been reported to interfere with lymphocyte function. However, rose hip and GOPO exhibit strong antioxidative properties and were reported to preserve cell membranes from disintegration.

Thus, the ingredients in the combinations of rose hip and fish oil, GOPO and fish oil, GOPO, fish oil and rose hip are complementary to each other and thus superior to fish oil or rose hip alone regarding the reduction of pain and stiffness in patients suffering from osteoarthrosis, as established by the above examples, and further may have prophylactic effects to prevent further disease progression.

These results are quite surprising particularly in view of the concentrations used. To treat inflammation as described in the '960 patent, about 45 g were used on a daily basis as opposed to the 0.01-500 mg GOPO per day and optionally up to 5 g rose hip concentrate per day utilized in the present combination. Fish oil is typically consumed at about 1 to 4 g per day and this is higher than the dosages that may be used in accordance with the present invention of about 0.1 to 0.8 g. Despite the much lower concentrations, a significantly greater beneficial result is obtained which reduces joint pain and joint stiffness, and these results are believed to not only being beneficial by the treatment of the joint symptoms, but, in view of the likely pathways of action, may also be utilized as a prophylaxis for preventing or limiting progression of joint disease.

While preferred embodiments of the present invention have been shown and described, it will be understood by those skilled in the art that various changes or modifications may be made without departing from the scope of the present invention.

1. A composition comprising:
a glycoside of a mono- or diacylglycerol compound selected from the group consisting of 3-β-D-galactopyranosyloxy-2-(octadeca-9Z,12Z,15Z-trienoyloxy)propanyl octadeca-9Z,12Z,15Z-trienoate, 3-β-D-glucopyranosyloxy-2-(octadeca-9Z,12Z,15Z-trienoyloxy)propanyl octadeca-9Z,12Z,15Z-trienoate, 3-α-D-galactopyranosyloxy-2-(octadeca-9Z,12Z,15Z-trienoyloxy)propanyl octadeca-9Z,12Z,15Z-trienoate, and 3-α-D-glu-copyranosyloxy-2-(octadeca-9Z,12Z,15Z-trienoyloxy)propanyl octadeca-9Z,12Z,15Z-trienoate, 6-O-(α-D-galactopyranosyl)-3-β-D-galactopyranosyloxy-2-(octadeca-9Z,12Z,15Z-trienoyloxy)propanyl octadeca-9Z,12Z,15Z-trienoate and 6-O-(α-D-galactopyranosyl)-3-α-D-galactopyranosyloxy-2-(octadeca-9Z,12Z,15Z-trienoyloxy)propanyl octadeca-9Z,12Z,15Z-trienoate and mixtures thereof, and,
an oil rich in n-3 polyunsaturated fatty acids selected from the group consisting of fish oil, shark oil, whale oil, seal oil, linseed oil, chia seed oil, kiwifruit seed oil, perilla seed oil, cowberry seed oil and mixtures thereof.

2. The composition according to claim 1, wherein the mono- or diacylglycerol compound is 3-β-D-galactopyranosyloxy-2-(octadeca-9Z,12Z,15Z-trienoyloxy)propanyl octadeca-9Z,12Z,15Z-trienoate.

3. The composition according to claim 1, wherein the oil rich in n-3 polyunsaturated fatty acids is fish oil.

4. The composition according to claim 2, wherein the oil rich in n-3 polyunsaturated fatty acids is fish oil.

5. The composition according to claim 1 further comprising a physiologically acceptable carrier.

6. The composition according to claim 1, wherein the formulation is in a unit dosage form.

7. The composition according to claim 3, wherein the fish oil is in a powdered form containing about 41 to 42.2 mg/g EPA as triglycerides and about 27 to 28.8 mg/g DHA as triglycerides.

8. The composition according to claim 4, wherein the fish oil is in a powdered form containing about 41 to 42.2 mg/g EPA as triglycerides and about 27 to 28.8 mg/g DHA as triglycerides.

9. The composition according to claim 1, wherein the composition is in the form of a daily dose form applicable for administration of fish oil in a daily dose corresponding to about 0.1 g to about 1.8 g of n-3 polyunsaturated fatty acids and about 0.01-500 mg of the glycoside of a mono- or diacylglycerol compound in unitary or multiple doses.

10. The composition according to claim 1, further comprising a rose hip concentrate or an extract from rose hips from the Rose family such as Rosa canina (“dog rose-hip”), Rosa gallica, Rosa condita, Rosa rugosa, Rosa hugonis, Rosa nitida, Rosa pendulina, Rosa pimpinellifolia, and/or Rosa sericea.

11. The composition according to claim 3, further comprising a rose hip concentrate or an extract from rose hips from the Rose family such as Rosa canina (“dog rose-hip”), Rosa gallica, Rosa condita, Rosa rugosa, Rosa hugonis, Rosa nitida, Rosa pendulina, Rosa pimpinellifolia, and/or Rosa sericea.

12. The composition according to claim 4, further comprising a rose hip concentrate or an extract from rose hips from the Rose family such as Rosa canina (“dog rose-hip”), Rosa gallica, Rosa condita, Rosa rugosa, Rosa hugonis, Rosa nitida, Rosa pendulina, Rosa pimpinellifolia, and/or Rosa sericea.

13. The composition according to claim 10, wherein the composition is in the form of a daily dose applicable for administration of fish oil in a daily dose corresponding to about 0.1 g to about 1.8 g of n-3 polyunsaturated fatty acids, about 0.01-500 mg of the glycoside of a mono- or diacylglycerol compound and about 2.5-6 g rosehip extract in unitary or multiple doses.

14. The composition according to claim 11, wherein the composition is in the form of a daily dose applicable for administration of fish oil in a daily dose corresponding to about 0.1 g to about 1.8 g of n-3 polyunsaturated fatty acids, about 0.01-500 mg of the glycoside of a mono- or diacylglycerol compound and about 2.5-6 g rosehip extract in unitary or multiple doses.

15. The composition according to claim 12, wherein the composition is in the form of a daily dose applicable for administration of fish oil in a daily dose corresponding to about 0.1 g to about 1.8 g of n-3 polyunsaturated fatty acids, about 0.01-500 mg of the glycoside of a mono- or diacylglycerol compound and about 2.5-6 g rosehip extract in unitary or multiple doses.

16. A composition according to claim 10, wherein the composition is in the form of a daily dose applicable for administration of fish oil in a daily dose corresponding to about 0.1 g to about 1.8 g of n-3 polyunsaturated fatty acids, about 0.01-500 mg of the glycoside of a mono- or diacylglycerol compound and about 1-3 g rose-hip extract in unitary or multiple doses.

17. A composition according to claim 11, wherein the composition is in the form of a daily dose applicable for administration of fish oil in a daily dose corresponding to about 0.1 g to about 1.8 g of n-3 polyunsaturated fatty acids, about 0.01-500 mg of the glycoside of a mono- or diacylglycerol compound and about 1-3 g rose-hip extract in unitary or multiple doses.

18. A composition according to claim 12, wherein the composition is in the form of a daily dose applicable for administration of fish oil in a daily dose corresponding to about 0.1 g to about 1.8 g of n-3 polyunsaturated fatty acids, about 0.01-500 mg of the glycoside of a mono- or diacylglycerol compound and about 1-3 g rose-hip extract in unitary or multiple doses.

19. A method for the treatment, alleviation or prophylaxis of inflammatory conditions in a mammal comprising:
administering to the mammal an effective amount of a composition comprising a glycoside of a mono- or diacylglycerol compound and an oil rich in n-3 polyunsaturated fatty acids, wherein the glycoside of a mono- or diacylglycerol compound is selected from 3-β-D-galactopyranosyloxy-2-(octadeca-9Z,12Z,15Z-trienoyloxy)propanyl octadeca-9Z,12Z,15Z-trienoate, 3-β-D-glucopyranosyloxy-2-(octadeca-9Z,12Z,15Z-trienoyloxy)propanyl octadeca-9Z,12Z,15Z-trienoate, 3-α-D-galactopyranosyloxy-2-(octadeca-9Z,12Z,15Z-trienoyloxy)propanyl octadeca-9Z,12Z,15Z-trienoate, and 3-α-D-glucopyranosyloxy-2-(octadeca-9Z,12Z,15Z-trienoyloxy)propanyl octadeca-9Z,12Z,15Z-trienoate, 6-62 -(α-D-galactopyranosyl)-3-β-D-galactopyranosyloxy-2-(octadeca-9Z,12Z,15Z-trienoyloxy)propanyl octadeca-9Z,12Z,15Z-trienoate and 6-O-(α-D-galactopyranosyl)-3-α-D-galactopyranosyloxy-2-(octadeca-9Z,12Z,15Z-trienoyloxy)propanyl octadeca-9Z,12Z,15Z-trienoate and mixtures thereof, and wherein the oil rich in n-3 polyunsaturated fatty acids is selected from a group comprising fish oil, shark oil, whale oil, seal oil, linseed oil, chia seed oil, kiwifruit seed oil, perilla seed oil, cowberry seed oil and mixtures thereof.

20. The method of claim 19, wherein the inflammatory condition is selected from hepatitis, meningitis, rheumatoid arthritis, inflammatory bowl diseases, such as Crohns disease, allergic syndromes, diabetes, congestive heart disease, psoriatic, reactive or osteo-arthritis or other arthritides or joint diseases, multiple sclerosis, atherosclerosis, sepsis/septic shock, dermal inflammation, graft rejection, and inflammation secondary to chemotherapy or radiotherapy of neoplastic diseases.

21. The method of claim 19, wherein the mammal is selected from the group consisting of humans, horses, dogs, cats, sheep, pigs and cattle.

22. A method of alleviating the symptoms associated with an inflammatory condition in a mammal comprising:
providing a composition comprising a glycoside of a mono- or diacylglycerol compound and an oil rich in n-3 polyunsaturated fatty acids, wherein the glycoside of a mono- or diacylglycerol compound is selected from 3-β-D-galactopyranosyloxy-2-(octadeca-9Z,12Z,15Z-trienoyloxy)propanyl octadeca-9Z,12Z,15Z-trienoate, 3-β-D-gluco-pyranosyloxy-2-(octadeca-9Z,12Z,15Z-trienoyloxy)propanyl octadeca-9Z,12Z,15Z-trienoate, 3-α-D-galactopyranosyloxy-2-(octadeca-9Z,12Z,15Z-trienoyloxy)propanyl octadeca-9Z,12Z,15Z-trienoate, and 3-α-D-glucopyranosyloxy-2-(octadeca-9Z,12Z,15Z-trienoyloxy)propanyl octadeca-9Z,12Z,15Z-trienoate, 6-O-(α-D-galactopyranosyl)-3-β-D-galactopyranosyloxy-2-(octadeca-9Z,12Z,15Z-trienoyloxy)propanyl octadeca-9Z,12Z,15Z-trienoate and 6-O-(α-D-galactopyranosyl)-3-α-D-galactopyranosyloxy-2-(octadeca-9Z,12Z,15Z-trienoyloxy)propanyl octadeca-9Z,12Z,15Z-trienoate and mixtures thereof, and wherein the oil rich in n-3 polyunsaturated fatty acids is selected from a group comprising fish oil, shark oil, whale oil, seal oil, linseed oil, chia seed oil, kiwifruit seed oil, perilla seed oil, cowberry seed oil and mixtures thereof, and,
administering a therapeutically effective amount of the composition to the mammal.

23. The method according to claim 22, wherein the inflammatory condition is selected from the group consisting of hepatitis, meningitis, rheumatoid arthritis, inflammatory bowl diseases, such as Crohns disease, allergic syndromes, diabetes, congestive heart disease, psoriatic, reactive or osteo-arthritis or other arthritides or joint diseases, multiple sclerosis, atherosclerosis, sepsis/septic shock, dermal inflammation, graft rejection, and inflammation secondary to chemotherapy and radiotherapy of neoplastic diseases.

24. The method according to claim 22, wherein the mammal is selected from the group consisting of humans, horses, dogs, cats, sheep, pigs and cattle.
